(19) United States
(12) Patent Application Publication
(10) Pub. No.: US 2005/0131434 A1
(43) Pub. Date: Jun. 16, 2005

(54) Reciprocating instrument for separating a cornea to form a corneal flap

(76) Inventor: Gholam A. Peyman, New Orleans, LA (US)

Correspondence Address: ROYLANCE, ABRAMS, BERDO & GOODMAN, L.L.P., 1300 19TH STREET, N.W., SUITE 600, WASHINGTON,, DC, 20036, US

(21) Appl. No.: 10/733,441

(22) Filed: Dec. 12, 2003

(51) Int. Cl. A61F 9/00

(52) U.S. Cl. 606/166

(57) ABSTRACT

The present invention relates to a device for separating the cornea of the eye and in particularly the cornea and a method for using the same. The device includes a head portion adapted to reciprocate, and a wire attached to the head portion. The wire is adapted to separate the cornea into first and second surfaces, which form a flap in the cornea of the eye.

### FIELD OF THE INVENTION

The present invention relates to an instrument for separating the cornea of an eye to form a flap in the surface of the cornea. More particularly, the present invention relates to an instrument that has a reciprocating wire that is adapted to separate an epithelial layer from the remainder of the cornea to form an epithelial flap in the surface of the cornea.

### BACKGROUND OF THE INVENTION

The cornea consists of three regions, the epithelial sheet, the stroma, and the endothelial lining. The epithelial sheet is the outermost region. It consists of between five to eight layers of cells and makes up approximately 10% of the thickness of the cornea. Moreover, the epithelial sheet is renewable, in other words, capable of re-growth. In addition, the epithelial sheet is filled with thousands of tiny nerve endings, making the cornea extremely sensitive to pain when the epithelial sheet is, for example, scratched. The stroma is the middle region. It is located behind the epithelial sheet and makes up approximately 90% of the thickness of the stroma. The endothelial lining is the innermost region. It is a single layer of cells located behind the stroma.

During refractive eye surgery, the shape of the stroma is changed. For example, in Photo Refractive Keratectomy (“PRK”) the shape of the stroma is changed with an excimer laser. First, however, the cells in the epithelial sheet are killed or removed using a laser, a chemical, or a scraping device. After the PRK, the epithelial sheet grows back over the stroma. However, during this time period, the patient may experience pain and/or poor vision. In addition, regression might occur. Regression is the growth of the epithelial sheet in a pattern which restores, or nearly restores, the shape of the cornea prior to the PRK.

In Laser Assisted In Situ Keratomileusis (“LASIK”), the shape of the stroma is also changed using an excimer laser. In LASIK, a microkeratome is used to hinge back the outermost 20-30% of the cornea. The excimer laser is then used to change the shape of the exposed stroma. Because LASIK maintains the epithelial sheet, LASIK tends to avoid the problems discussed above in regard to PRK. However, LASIK is dependent on the use of the microkeratome, which may jam, shred, or lose the corneal flap. Moreover, a suction device must be used in conjunction with the microkeratome, increasing intra ocular pressure up to approximately 100 mm Hg. For some vulnerable patients, the increase in intra ocular pressure can harm their eyes.

In Laser Epithelial Keratomileusis (“LASEK”), the epithelial sheet is loosened with an alcohol solution, then rolled back to expose the stroma. The excimer laser is then used to change the shape of the stroma and the loosened epithelial sheet is repositioned over the stroma. However, in LASEK, the patient experiences a slow return to clear vision and must wear a contact lens on the affected eye for a number of days. The slow return to clear vision is due to the use of the alcohol solution, which kills some of the epithelial cells. Moreover, the presence of dead epithelial cells renders the cornea vulnerable to infection, a situation that is enhanced because of the post-operative use of a contact lens.

Additional devices have been developed to separate the epithelial sheet. For example, a subepithelial separator, which is a microkeratome-based device, uses a blunt blade and low suction to mechanically separate a hinged epithelial sheet without alcohol. The sheet is then reflected nasally onto a contact lens. After ablation of the exposed cornea, the sheet is replaced along with a contact lens. This type of device is preferably to the use of alcohol, since the mechanical separation takes place under the basement membrane, thus preserving the integrity of the epithelial flap.

However, with a conventional subepithelial separator, the use of a small knife or blade is generally required. It is very difficult to maintain the proper blade sharpness with these types of blades. If the blade is too sharp or not sharp enough, it is difficult to predict the type of cut that will result and whether or not the blade will cut deep enough or too deep. It is generally very important that the stroma is not cut during this type of procedure. Additionally, blades can be very expensive to purchase and maintain.

Accordingly, a need exists for an improved instrument for forming an epithelial flap.

### SUMMARY OF THE INVENTION

Accordingly, it is an object of the present invention to provide an instrument adapted to form a flap in the surface of the cornea.

Another object of the present invention is to provide a reciprocating instrument adapted to form an epithelial flap in the surface of the cornea of the eye.

Yet another object of the present invention is to provide an instrument that has a reciprocating wire that is adapted to form an epithelial flap in the surface of the cornea of the eye.

The foregoing objects are basically attained by a device for forming a flap on the surface of a cornea of an eye, including a head portion adapted to reciprocate, and a wire attached to the head portion and adapted to separate the cornea into first and second surfaces, which form a flap in the cornea of the eye.

The foregoing objects are further attained by a device for forming a flap in the surface of a cornea of an eye, including first member having a head portion, with a reciprocating member coupled to the head portion. A first arm and a second arm are coupled to the reciprocating member, and a wire extends between the first and second arm and are adapted to separate the cornea into first and second surfaces.

By forming an instrument for forming epithelial flaps in this manner, precise epithelial flaps that remain intact can be inexpensively and effectively formed.

Other objects, advantages, and salient features of the present invention will become apparent to those skilled in the art from the following detailed description, which, taken in conjunction with the annexed drawings, discloses preferred embodiments of the invention.

### BRIEF DESCRIPTION OF THE DRAWINGS

Referring to the drawings which form a part of this disclosure:

FIG. 1 illustrates a side elevational view of the preferred embodiment of the present invention, including the reciprocating member and the positioning member coupled together and positioned relative the eye of a patient;

FIG. 2 is a top view of the present invention of FIG. 1;

FIG. 3 is a side view in section of the present invention forming an epithelial flap in the surface of the cornea of an eye;

FIG. 4 is a side elevational view of the eye of FIG. 3 with an epithelial flap formed therein;

FIG. 5 is a bottom view of the present invention, including the reciprocating member and the positioning member coupled together;

FIG. 6 is a top perspective view of the reciprocating member and the positioning member of FIG. 5 detached from one another;

FIG. 7 is a partial top view of the positioning member of FIG. 5;

FIG. 8 is a partial side view of the positioning member of FIG. 7;

FIG. 9 is a partial bottom view of the positioning member of FIG. 8;

FIG. 10 is a partial front view of the positioning member of FIG. 9;

FIG. 11 is a partial top perspective of the positioning member of FIG. 10;

FIG. 12 is a partial bottom view of the reciprocating member of FIG. 5;

FIG. 13 is a partial top perspective view of the reciprocating member of FIG. 12;

FIG. 14 is a partial side view of the reciprocating member of FIG. 13;

FIG. 15 is a partial side perspective view of the view of the reciprocating member of FIG. 14;

FIG. 16 is a partial top perspective of the positioning member of FIG. 15.

FIG. 17 is a side view of a second embodiment of the present invention, wherein the positioning member is coupled or affixed to the reciprocating member;

FIG. 18 is a third embodiment of the present invention, wherein a blank or lens is positioned overlying the epithelial flap;

FIG. 19 is a bottom perspective view of a cutting device used to form a groove in the cornea;

FIG. 20 is a side view of the cutting device of FIG. 19 being applied to surface of the cornea; and

FIG. 21 is a top view of the cornea of the eye of FIG. 19, showing the groove formed by the cutting device.

### DETAILED DESCRIPTION OF THE INVENTION

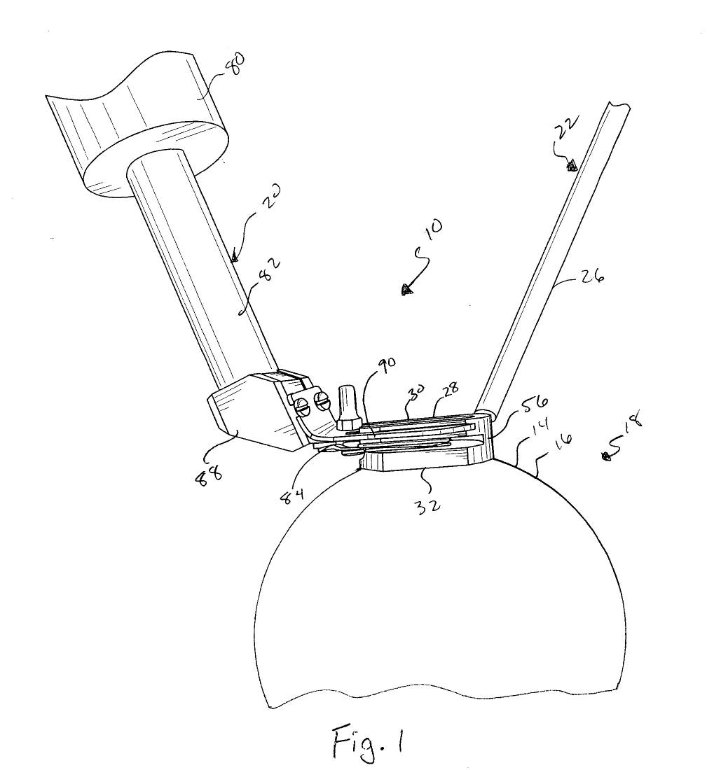

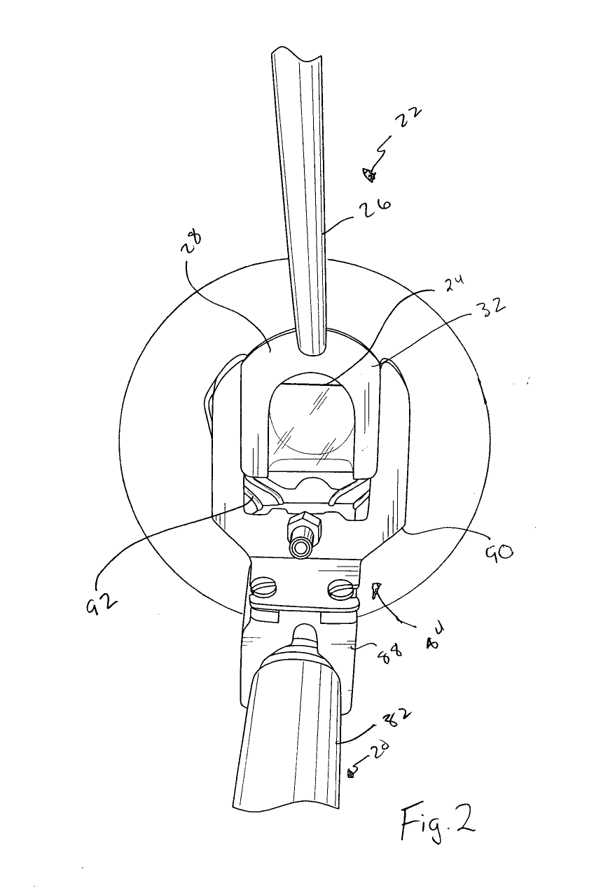

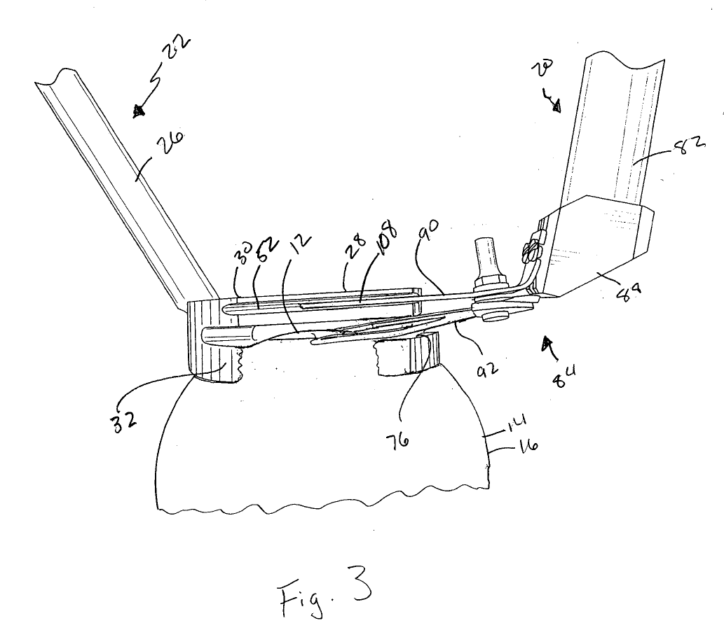

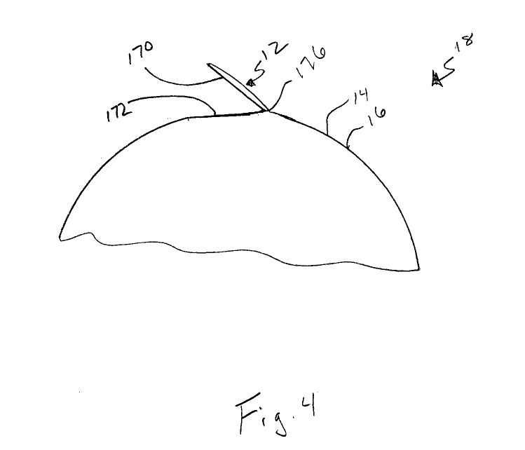

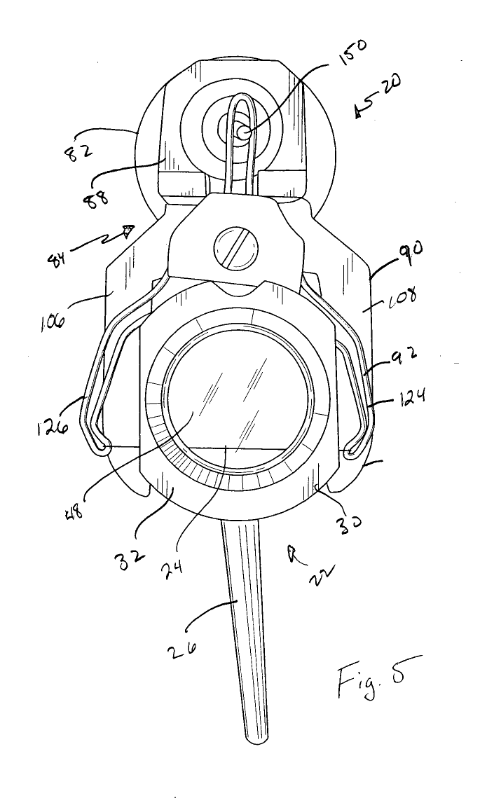

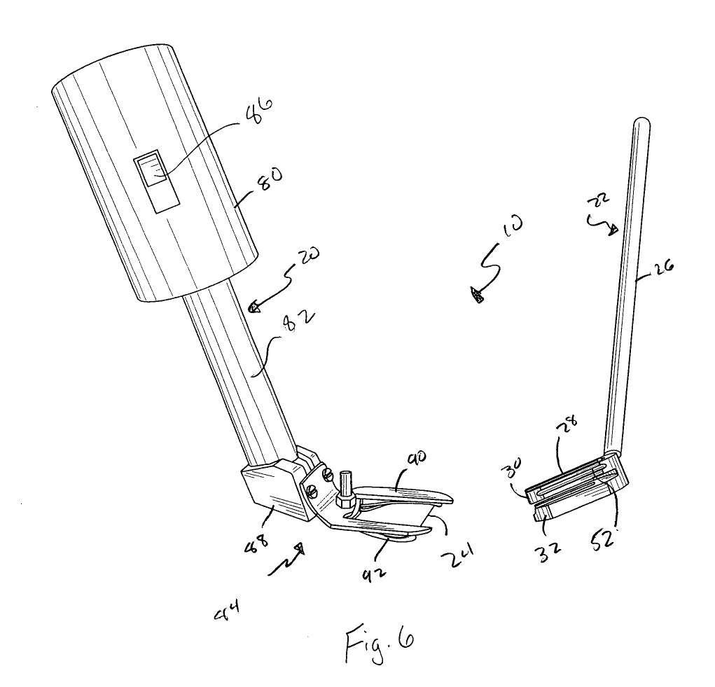

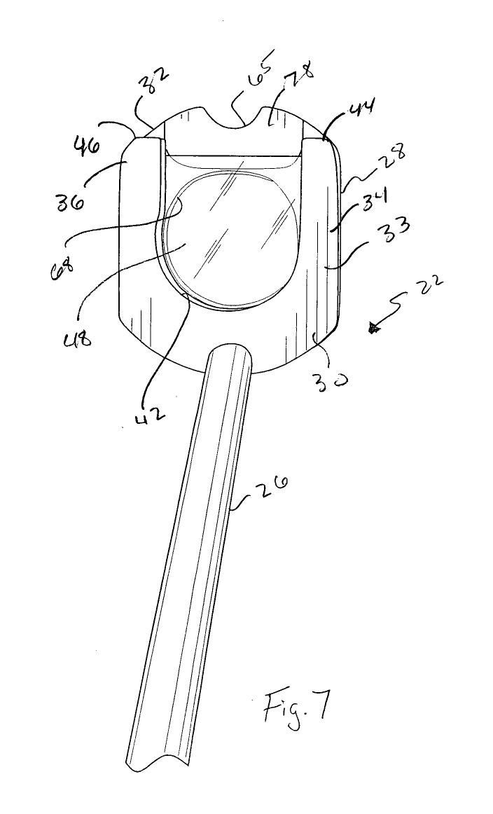

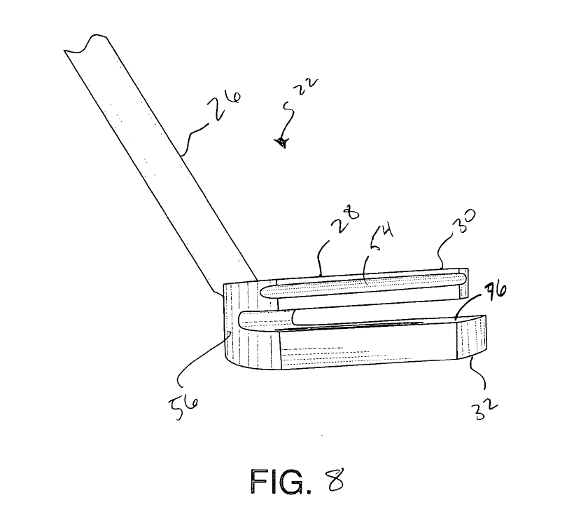

As illustrated in FIGS. 1-6 the present invention relates to an instrument 10 for forming an epithelial flap 12 in the surface 14 of the cornea 16 of an eye 18. Instrument 10 includes a reciprocating or first member 20 and a positioning or second member 22, which couple together. The positioning member is adapted to be positioned on the surface 14 of the cornea 16 and position the reciprocating member 20 an appropriate distance from the surface of the cornea, when the positioning member and the reciprocating member are coupled together. Once the reciprocating member and the positioning member are coupled together and properly positioned on the eye, the reciprocating wire 24 is activated and the reciprocating member 20 is moved relative to the positioning member 22 (FIG. 3), with the wire 24 reciprocating or cutting through the surface 14 of the cornea 16 and forming the desired flap 12.

As shown in FIGS. 7-11, the positioning member 22 has a handle 26 and a head portion 28. Handle 26 is preferably a metal cylindrical handle that is coupled to head portion 28. Preferably handle 26 is coupled to head portion 28 using a welded joint, but can be coupled thereto using in any conventional method, such as threads, friction or glue.

Head portion 28 is preferably formed from metal or any other suitable material and has an upper portion 30 and a lower portion 32. Upper portion 30 is preferably substantially U-shaped with a first arm 34, a second arm 36 and a base portion 38. The handle is preferably coupled to the base portion 38; however, the handle can be coupled to any suitable portion of either the upper or lower portions.

As shown in FIGS. 8, 10 and 11, a groove 40 extends along the interior surface of first arm 34, the interior surface of base portion 38 and the interior surface of the second arm 36. In other words, the groove 40 extends along the interior surface of the upper portion 30 from of first end 44 of first arm 34 to the first end 46 of the second arm 36. Preferably, transparent member 48 is sized and configured to frictionally fit within groove 40. Transparent member 48 is preferably formed from plastic; however it can be formed from any transparent material desired, such as glass or any other suitable material. Transparent member 48 has an exposed side or surface 50 at the open end of the U-shaped area of upper portion 30, or in other words, surface 50 extends from end 44 to end 46. However, it is noted that it is not necessary to have a transparent member 48 positioned within groove 40. The U-shaped area can have no material extending thereacross, and be completely open, if desired.

As shown in FIGS. 8, 10 and 11, arms 34 and 36 have grooves 52 and 54 extending along the exterior portion thereof. Grooves 52 and 54 preferably extend substantially along the length of a respective arm and are sized and configured to receive the reciprocating member.

Lower portion 32 is preferably unitary with upper portion and connected thereto by portion 56, as shown in FIG. 8. Lower portion 32 preferably has an arcuate or rounded front or first end 58 and an arcuate or rounded second end 60. Connecting first end 58 and second end 60 are substantially straight sides 62 and 64. Sides 62 and 64 are preferably substantially parallel. First end 58 has a substantially arcuate area 65 removed therefrom. Centered in the middle of lower portion 32 is opening 66. Opening 66 is preferably centered under and underlying transparent member 48. Opening 66 is defined by surface 68, which is adjacent sloped or angled surface 70. Sloped surface 70 is adjacent another sloped surface 72, which terminates at bottom surface 74, which is a substantially flat, planar surface. Opening 66 is defined by these sloped surfaces to allow easy access of the cornea through the opening and, so that a portion of the surface of the cornea extends through the opening, as shown in FIG. 3.

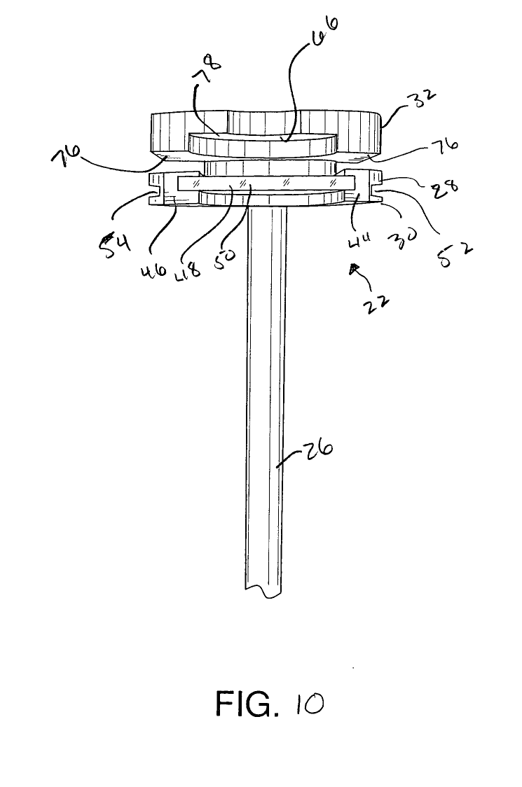

As shown in FIGS. 10 and 11, lower portion 32 has an upper surface 76 that is substantially planar and positioned apart from the lower surface 35 of the upper portion 30. Additionally, lower portion 32 has a recessed area 78.

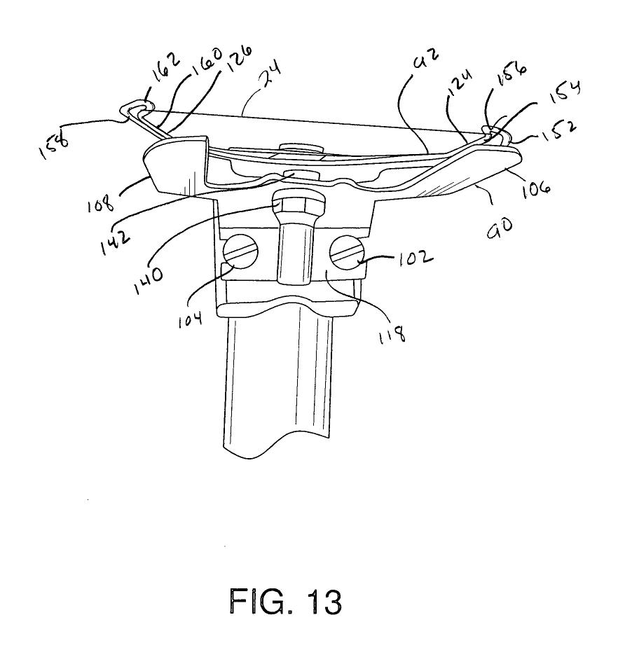

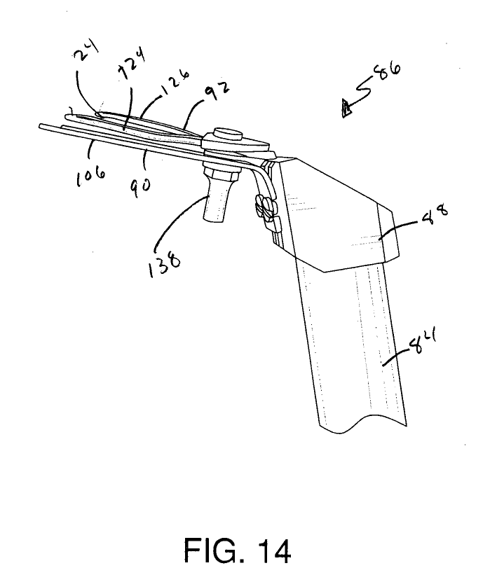

As shown in FIGS. 6 and 12-16, reciprocating member 20 preferably is formed from plastic (or other suitable material) and has a body or handle portion 80, neck portion 82 and a head portion 84. Handle portion 80 preferably is a cylindrical housing for an electric motor (not shown) and a battery source (not shown); however, handle portion 80 can merely be a gripping device with the motor and power source external thereto. Handle portion 80 also includes an on/off switch 86, which turns on and off the electric motor. Neck portion 84 is preferably substantially cylindrical and connects the head portion 84 and the handle portion 80, and preferably houses a shaft to connect the electric motor with the reciprocating head.

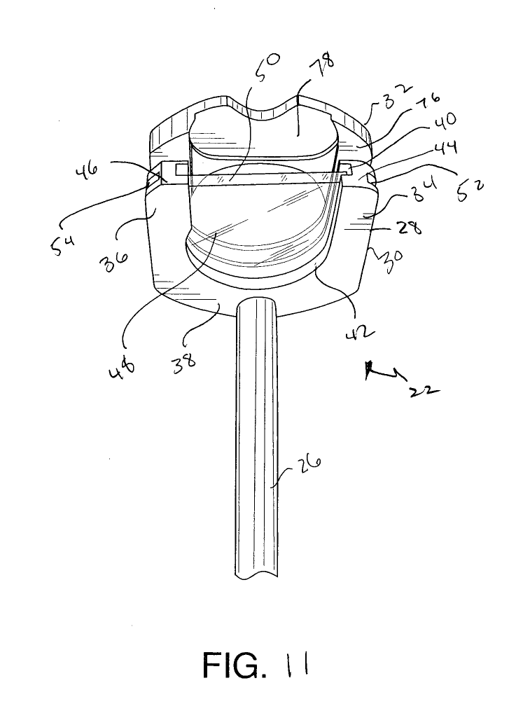

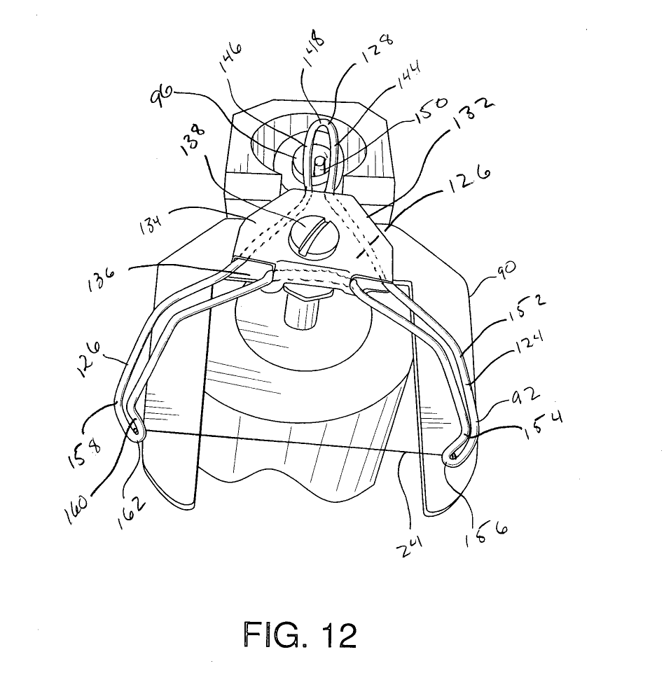

As shown in FIGS. 12-16, head portion 86 includes a cap 88, a guard or attachment portion 90 and a reciprocating member 92. Cap 88 is preferably formed of plastic and facilitates the connection of guard 90 and reciprocating member 92 to the neck and body of the reciprocating member 20. Cap 88 has a recessed portion 94 that accommodates the end 96 of the shaft (not shown) and end 98 of reciprocating member 92. Additionally, cap 88 has a front substantially flat surface 100 that receives screws or fasteners 102 and 104 that couple the guard 90 thereto.

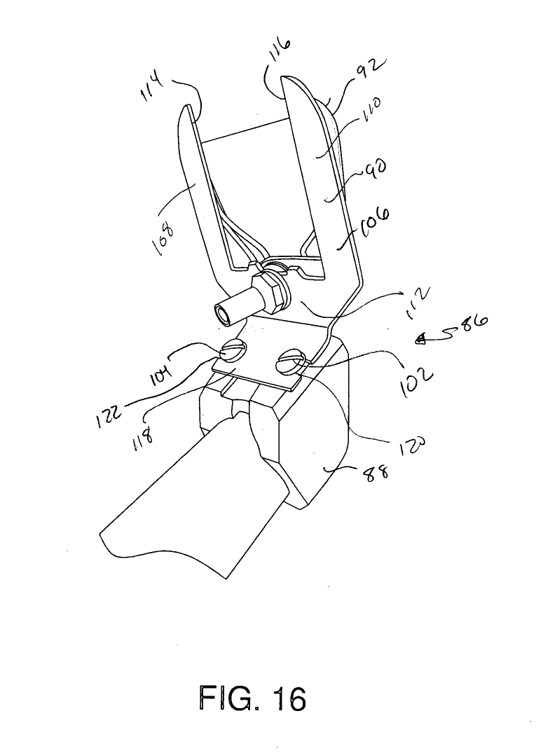

Guard 90 is preferably formed of metal and has a substantially U-shaped portion 106, as shown in FIG. 16. U-shaped portion 106 has a first arm 108, a second arm 110 and a base portion 112. First and second arms 108 and 110 are preferably substantially parallel and each extends at substantially 90° from the base portion 112. Each arm 108 and 110 has an inner surface 114 and 116, respectively, that is substantially smooth and straight. Additionally, the arms 108 and 110 have a thickness and are spaced from each other in such a manner that they can be inserted into the grooves 52 and 54 on the first and second arms of the positioning member, respectively.

Extending from the U-shaped portion 106 is connecting portion 118. Connecting portion 118 has two openings or slots 120 and 122 to accommodate screws 102 and 104, respectively, thereby coupling the guard 90 to the cap 88.

As shown in FIG. 12, reciprocating member 92 is a metal wire frame and is substantially U-shaped or Y-shaped. Member 92 has a first arm 124, a second arm 126, a base portion 128 and a rear stem portion 130. Base portion 128 is the portion of the reciprocating member where the first arm 124 meets or joins with the second arm 126. Clamp member 132 couples reciprocating member 92 to the guard member 90.

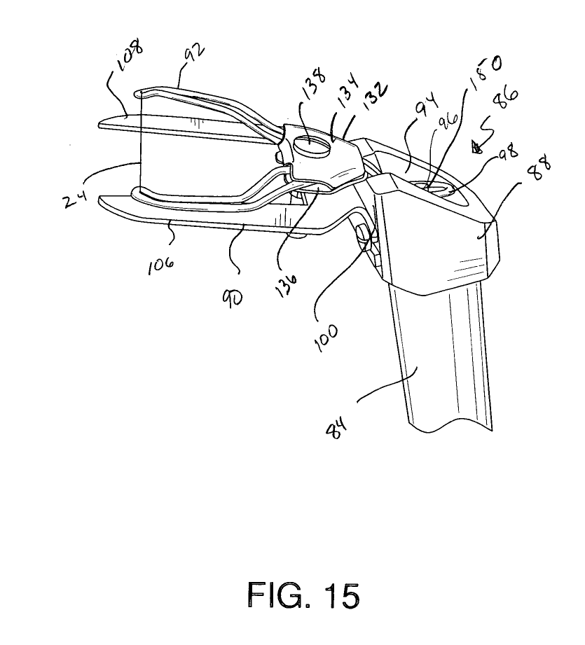

As shown in FIGS. 12 and 15, clamp member 132 is generally triangular and includes an upper clamp 134 and a lower clamp 136. Upper clamp 134 overlies the reciprocating member 132 at approximately the base portion and has an opening therein that is adapted to receive a screw 138. Lower clamp 136 is positioned between the reciprocating member 92 and the guard 90 and is substantially the same size and shape as the upper clamp, i.e., generally triangular. Lower clamp also has an opening therein to receive screw 138.

Screw 138 extends through the openings in the upper and lower clamps and into the guard, as shown in FIGS. 15 and 16. Nut 140 couples to the screw 138 and is adapted to couple the reciprocating member 92 to the guard 90. A bushing 142 is positioning around the screw 138 and is adapted to properly position the reciprocating member and reduce the friction to allow the reciprocating member to reciprocate freely.

As shown in FIGS. 12 and 15, rear stem portion 130 has a first wire portion 144 and a second wire portion 146 that are coupled together at the end 148 of the rear stem portion. As shown in FIG. 12, reciprocating drive 150 is coupled to end 96 of the drive shaft and extends between the first wire portion and the second wire portion. The drive 150 is preferably a cylindrical projection that extends upwardly from end 96; however, dive 150 can be any shape or configuration suitable to cause the reciprocating member to oscillate back and forth. Drive 150 preferably extends upwardly from the periphery of the drive shaft, preferably substantially parallel to the axis of the drive shaft. The end 96 of the drive shaft is adapted to rotate 360° about the axis of the drive shaft, and thereby rotate the drive 150 in a 360° circle about the axis of the drive shaft. This rotation causes the reciprocating member 92 to reciprocate about screw 138 when the drive 150 rotates.

First arm 124 is formed of a first wire portion 152 and a second wire portion 154 that couple or connect at first end 156. Second arm 126 is formed of a third wire portion 158 and a fourth wire portion 160 and connect or couple at second end 162. Reciprocating wire 24 extends across the mouth of the U-shaped portion of the reciprocating member 92, i.e., wire 24 extends from first end 156 to second end 162. As shown in FIG. 15, wire 24 extends though first arm 124 between first wire portion 152 and second wire portion 154 and through second arm 126 between third wire portion 158 and fourth wire portion 160. Each end of wire 24 preferably is knotted and frictionally held between respective wire portions; however, wire 24 can be coupled to the arms of the reciprocating member in any suitable manner.

Reciprocating or cutting wire 24 is preferably substantially circular and formed from metal threads twined or twisted together. However, wire 24 can be any suitable material, such as single piece metal, surgical thread, TEFLON, DACRON, plastic, or any other material that would perform the desired cutting action. Additionally, the wire 24 does not necessarily need to be substantially circular and can be any suitable shape, such as substantially oval, substantially square, substantially rectangular, it can have a cutting edge or any other suitable shape. Furthermore, wire 24 has a diameter of preferably about 10 microns to about 500 microns and even more preferably about 50 microns to about 100 microns.

As noted above, preferably the wire 24 is reciprocated using an electric motor; however, the wire 24 can be reciprocated using an type of motor, electrical means, mechanical means or electro-mechanical means or any other means suitable, such as by hand. Additionally, wire 24 preferably oscillates back and forth at between a rate of about 50 vibrations per second to a rate of about 50,000 vibrations per second, and more preferably between about 8,000 vibrations per second to about 10,000 vibrations per second. Also, wire 24 oscillates back and forth at a rate between about 3 mm per second (when oscillating in the faster ranges) to 0.5 mm per second (when oscillating in the slower ranges); however, the wire 24 can oscillate at any suitable speed and distance. Furthermore, the wire can oscillate or reciprocate in any direction desirable and does not need to reciprocate from side to side. For example, the wire 24 can oscillate forward and backward or a combination of forward and backward and side to side. Preferably when oscillating forward and backward, wire 24 oscillates at a rate about 1 mm per second.

### Preferable Operation

As shown in FIGS. 1-3, preferably positioning member 22 and reciprocating member 20 are coupled together by sliding the first and second arms 106, 108 of guard member 90 into the appropriate slots or grooves 52, 54 on the upper portion 30 of the positioning member 22. Recessed area 78 preferably accommodates the head of screw 138, and wire 24 is positioned between upper portion 30 and lower portion 32. Preferably wire 24 is adjacent or even touching surface 76 of lower portion 32, so that only a minimal amount of the surface of the cornea needs to extend through opening 66. However, it is noted that the wire 24 can be positioned in any suitable position relative to the surface 76 and can also be positioned variably relative to surface 76 if desired. By positioning the wire relative thereto, the operator can more easily select the amount of cornea that extends through the opening 66 and the amount of epithelial layer that is separated.

Positioning member 22 is then positioned relative to the surface 14 of the cornea 16 of the eye 18. Pressure can be applied to the positioning member 22 thereby allowing the appropriate amount of the corneal surface 14 to pass through opening 66 and allow a portion of the cornea 16 to extend through the opening 66 beyond surface 76.

Reciprocating member can then be turned on, activating the reciprocating wire. The reciprocating wire is moved relative to the positioning member, as shown in FIGS. 1-3. The first and second arms 106, 108 slide out of grooves 52, 54, respectively, as the reciprocating member 20 moves relative to the positioning member 22, and the surface 14 of the cornea l6. This relative movement allows smooth and straight movement of the reciprocating wire 168 relative to the eye. As the reciprocating member 20 moves relative to the eye 18, the reciprocating wire 168 cuts or shaves a portion of the epithelial layer away from the stromal layer.

Preferably the wire cuts or separates the epithelial layer and the Basal Laminer from the Bowman's membrane and stromal layer of the cornea. This separation is achieved by the cutting wire burrowing under the Basal Laminer and gently separating this layer from the stroma and the Bowman membrane. By inherent design the wire is sharp enough to cut through the surface of the cornea, but not into the stroma, and therefore, it is relatively easy to form the desired epithelial flap.

The wire separates the cornea 16 into a first layer 170 and a second layer 172, forming flap 12. The first layer faces in a posterior direction and the second layer faces in an anterior direction, relative to the eye. Preferably flap 12 remains coupled to the cornea by hinge 176; however, the flap can remain attached to the cornea in any suitable manner or can be removed completely from the cornea. To remove the flap completely, a device as described in copending application Ser. No. can be used. The contents of U.S. Pat. No. 6,551,307 and U.S. patent application Ser. No. 10/356,730, the entire contents of both of which are hereby incorporated by reference in their entirety.

Additionally, the flap 12 can remain attached to the cornea 16 by a portion that remains connected to the cornea at the main optical axis. Moving the reciprocating member 20 is a 360° arc about the main optical axis of the eye forms this type of flap. This arc can be achieved by hand or by having a positioning member that allows the reciprocating member 20 to be rotated thereabout.

While positioning member 22, described herein, is preferable, it is possible to design any type of positioning member that would adequately position the wire 24 relative to the surface of the cornea. Any such positioning member can be fixed to the reciprocating member or can be a separate piece of equipment.

### Embodiment of FIG. 17

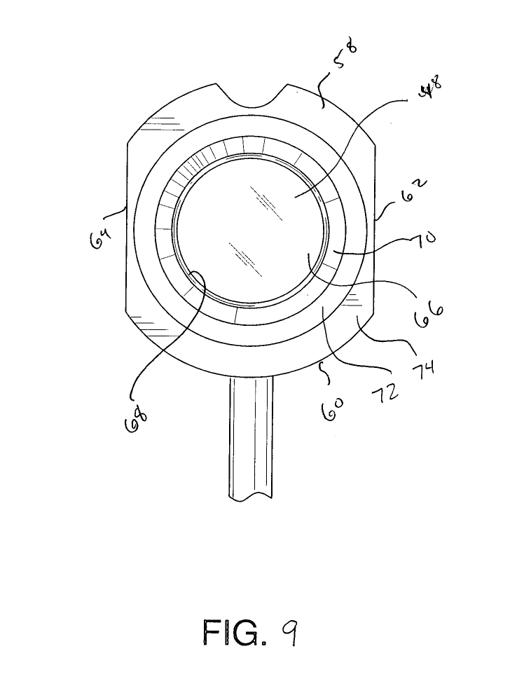

Additionally, as shown in FIG. 17, the instrument 10′ includes a positioning member 22′ that can be coupled and/or affixed to the reciprocating member 20′ by a connecting member 191. This instrument 10′ functions in a substantially similar manner as the instrument 10 in that the cornea extends through opening 66′ of the positioning member 22′ and is subsequently separated into first and second surfaces 170 and 172, respectively, or more preferably into a flap 12, as shown in FIG. 4.

However, the cutting wire 24′ preferably moves relative to the positioning member 22′ by movement of connecting portion 188′ which is coupled to a piston 190, which is in turn connected to head portion 88′. Piston 190 is adapted to move the reciprocating member 92′ and thus the cutting wire 24′ in a back and forth motion as indicated by arrow 192.

Any other description of instrument 10 is applicable to instrument 10′, as long as it is consistent with the specific description of FIG. 17, above. Additionally, any description of the reference numbers from the first embodiment of the present invention is applicable to the reference numbers used in FIG. 17.

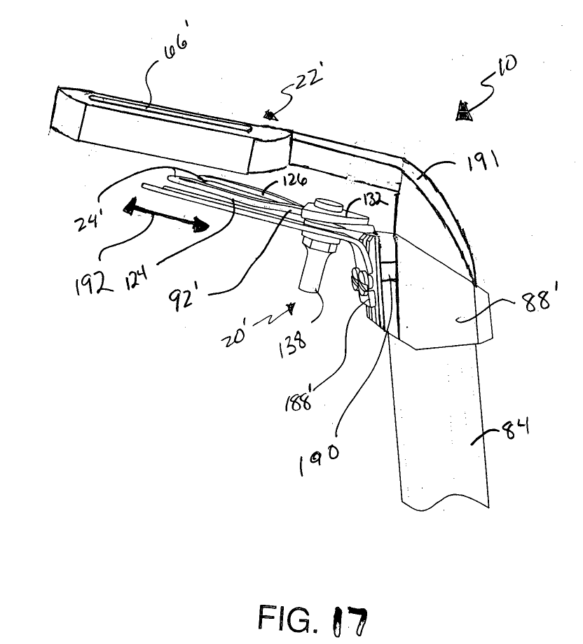

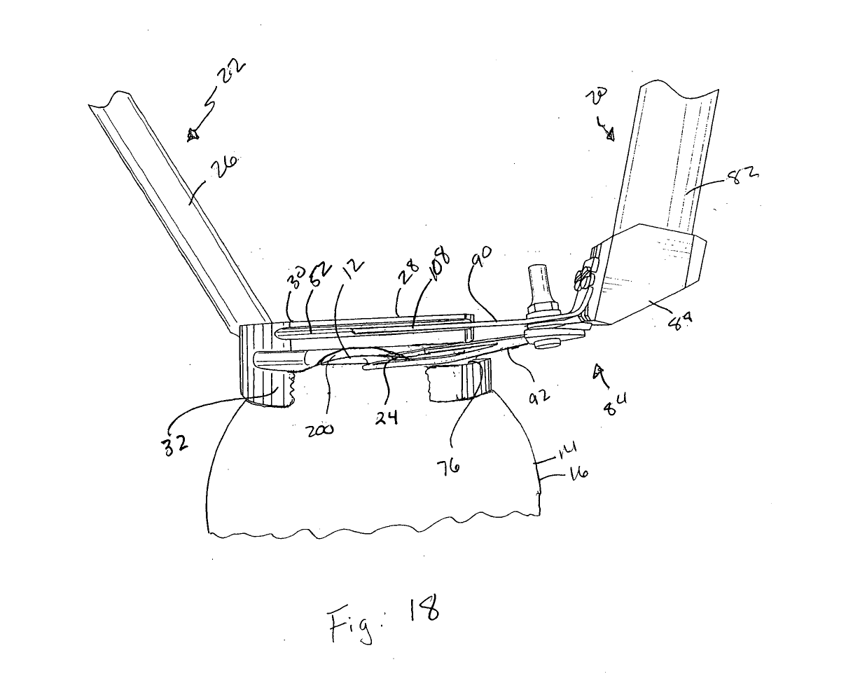

### Embodiment of FIG. 18

As shown in FIG. 18, a lens or blank 200 can be positioned overlying the surface of the cornea. Lens 200 allows the epithelial flap 12 to adhere thereto when the flap 12 is formed in the surface of the cornea. The flap can adhere to the lens using any suitable means such as a suctioning device or can adhere thereto merely by surface tension. Lens 200 can be any material suitable and is not necessarily transparent, let alone refractive. Lens 200 is merely a device that facilitates separation of the epithelial layer and Basal Laminer and can be any suitable material.

Flap 12 is formed using the lens 200 in the same manner as described above. Specifically, wire 24 separates the corneal surface into first and second corneal surfaces 170 and 172, respectively, by cutting or separating the epithelial cells and the Basal Laminer from the stroma of the cornea. The combination of the Basal Laminer and epithelial layer allows the flap to remain intact. Additionally having it adhere to the surface of the contact further facilitates the keeping the structural integrity of the flap intact. The flap can remain attached to the cornea, as described above, i.e., either at the periphery or substantially surrounding the main optical axis, or the flap can be completely separated from the cornea to be repositioned at a later time.

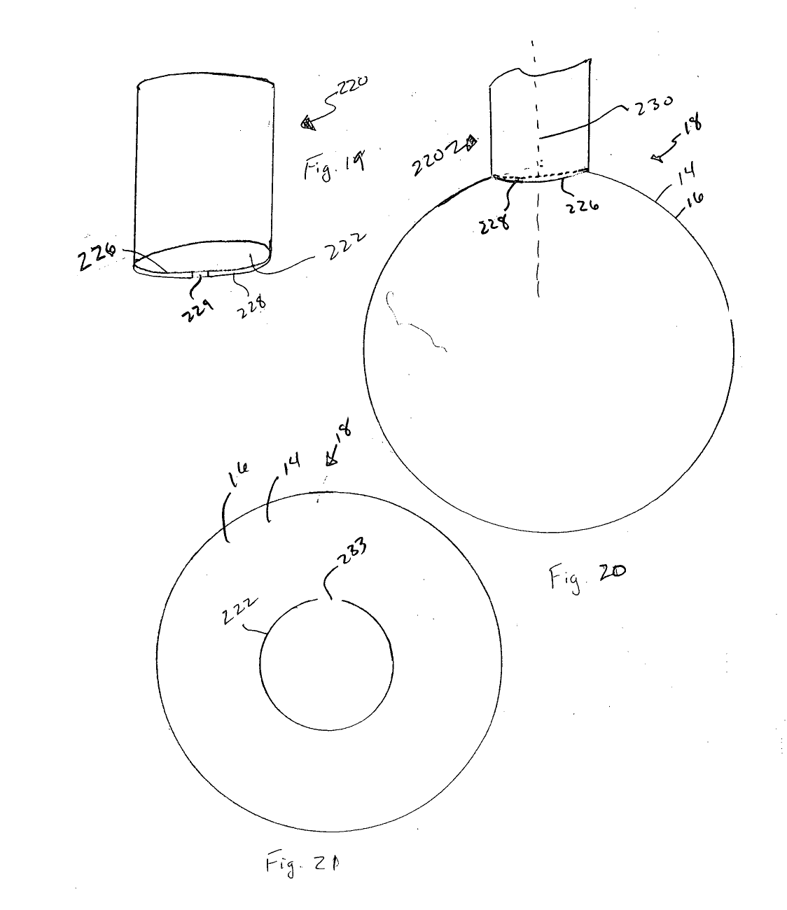

### Embodiment of FIGS. 19-21

As shown in FIGS. 19-21, cutting device 220 can be used to form a groove 222 in the surface of the cornea in the eye. Device 220 is preferably formed of metal or plastic; however, device 220 can be formed from any suitable material. The device 220 is preferably substantially cylindrical with a substantially flat, circular surface 224. Extending substantially around the periphery 226 of the surface 224 is a cutting device or blade 228.

Blade 228 extends preferably about 30 to about 130 microns in a direction substantially perpendicular to and away from planar surface 224. Furthermore, blade 228 is arcuate and preferably extends about 350° around the periphery of the surface 224; however, it is noted that the blade can extend 360° around the periphery of the surface or less than 360°. For example the blade can extend in an arc of about 180° or less to about 360° or less, if desired. This design creates a gap 229 at the periphery 226 in the blade 228.

Using device 220, surface 224 is positioned adjacent the surface of the cornea so that the main optical axis 230 is in about the center of surface 224. As sufficient force or pressure is applied to the to device 220 in a direction substantially parallel to the main optical axis, blade 228 cuts through the corneal surface and into the stromal layer, forming a groove 222. However, since the blade has a gap 229, the cornea groove 222 does not extend in a full circle or 360°, thus forming a connected or hinge portion 233. The groove extends about an arc that is about the same as the blade, or in other words in an arc of about 350°. However, as with the blade 228, the groove 222 can extend at any arc desired.

Once the groove 222 is formed the device 10, described above, can be used to form a flap that has substantially the same configuration as the groove, where the hinge portion 233 allows the flap to remain attached to the cornea, forming a flap as described above in FIG. 4. However, the flap formed using device 220 will actually extend into and include a portion of the stroma at a depth of preferably about 30 microns to about 130 microns from the surface of the cornea; however, the flap can have any suitable thickness. For example, the flap can be less than 30 micros (for example, about 10 microns) or the flap can be greater than 130 microns, up to about 180 microns. The flap preferably has a substantially uniform thickness, since the device 10 described above generally cannot cut through the stromal layer and can only dissect in a direction substantially parallel to the surface of the cornea.

This procedure is preferable conventional methods for forming corneal flaps, since the flap formed by the present invention has a thickness of about 30 to about 130 microns. Due to limitations on the cutting blades, conventional flaps can only have a thickness of between about 130 microns to about 180 microns. This limitation on flap thickness limits the amount of cornea that can be ablated. Therefore, the maximum correction in an eye is about plus or minus 8-10 diopters. By forming a flap that is between about 30 microns to about 130 microns the change in refractive error can be up to about plus or minus 20 diopters.

Additionally, to facilitate the dissection of the stromal layer, the above-described cutting wire can have a serrated edge.

While preferred embodiments have been chosen to illustrate the invention, it will be understood by those skilled in the art that various changes and modifications can be made therein without departing from the scope of the invention as defined in the appended claims.

1. A device for separating a cornea of an eye, comprising:
a head portion adapted to reciprocate; and
a wire attached to said head portion and adapted to separate the cornea to expose first and second surfaces.

2. A device according to claim 1, wherein
said wire is adapted to separate said cornea into said first and second surfaces to form a flap in the cornea of the eye.

3. A device according to claim 1, wherein
said head portion has a first arm and a second arm; and
said wire extends between said first arm and said second arm

4. A device according to claim 3, wherein
said first and second arms extend substantially parallel.

5. A device according to claim 1, wherein
a positioning member adapted to couple to said head portion and is adapted to position said wire at an appropriate position relative to the surface of the cornea.

6. A device according to claim 5, wherein
said positioning member is affixed to said head portion.

7. A device according to claim 1, further comprising
a guard member coupled to said head portion and adapted to protect the cornea from undesired separation.

8. A device according to claim 7, wherein
said guard has first and second arms.

9. A device according to claim 6, wherein
said positioning member has an upper portion and a lower portion, said wire adapted to be positioned between said upper and lower portions.

10. A device according to claim 9, wherein
said positioning member has a first slot and a second slot on opposite sides thereof, said first and second slots being adapted to receive said first and second arms of said guard member, respectively.

11. A device according to claim 9, wherein
said lower surface has an opening therein adapted to allow a portion of the cornea to pass therethrough and contact said wire.

12. A device according to claim 11, wherein
said upper portion has a transparent portion that substantially overlies said opening.

13. A device according to claim 1, further comprising
an electric motor adapted to reciprocate said head portion

14. A device according to claim 1, further comprising
a blank adapted to overlie said first and second surfaces.

15. A device according to claim 1, further comprising
a blade adapted to form an arcuate groove in the surface of the cornea of the eye.

16. A device according to claim 15, wherein
said wire is adapted to separate the cornea the along said groove.

17. A device according to claim 15, wherein
said blade adapted to form said arcuate groove so that said groove extends about in an arc of abut 350°.

18. A device for separating a cornea of an eye, comprising:
first member having a head portion;
a reciprocating member coupled to said head portion;
a first arm and a second arm coupled to said reciprocating member; and
a wire extending between said first and second arm, and adapted to separate the cornea to expose first and second surfaces.

19. A device according to claim 17, further comprising
a second member adapted to position said wire relative to the cornea.

20. A device according to claim 19, wherein
said head portion has an attachment portion coupled thereto and adapted to attach said first member to said second member.

21. A device according to claim 20, wherein
said second member has a first portion with a first slot and said second portion with a second slot.

22. A device according to claim 21, wherein
said attachment portion includes a first arm adapted to fit in said first slot and a second arm adapted to fit in said second slot.

23. A device according to claim 19, wherein
said second member has an upper portion and a lower portion, said wire adapted to be positioned between said upper and lower portions.

24. A device according to claim 23, wherein
said lower surface has an opening therein adapted to allow a portion of the cornea to pass therethrough and contact said wire.

25. A device according to claim 24, wherein
said upper portion has a transparent portion that substantially overlies said opening.

26. A device according to claim 19, wherein
said second member is affixed to said first member.

27. A device according to claim 18, further comprising
an electric motor adapted to reciprocate said head portion

28. A device according to claim 18, further comprising
a blank adapted to overlie said first and second surfaces.

29. A device according to claim 18, further comprising
a cutting device adapted to form a groove in the surface of the cornea of the eye.

30. A device according to claim 29, wherein
said cutting device includes a substantially circular surface; and
a blade extends from a portion of the periphery of said substantially circular surface, substantially perpendicular from said substantially circular surface.

31. A device according to claim 30, wherein
said blade extends about 350° about said periphery.
